(19) United States
(12) Patent Application Publication
(10) Pub. No.: US 2013/0304096 A1
(43) Pub. Date: Nov. 14, 2013

(54) FASCIAL CLOSURE SUTURE DEVICE

(71) Applicant: The Johns Hopkins University, Baltimore, MD (US)

(72) Inventors: Hien Nguyen, Baltimore, MD (US); Sohail Zahid, Baltimore, MD (US); Ang Tu, Baltimore, MD (US); Daniel Peng, Baltimore, MD (US); Stephen Van Kooten, Baltimore, MD (US); Leslie Myint, Baltimore, MD (US); Anvesh Annadanam, Baltimore, MD (US); Luis Herrera, Baltimore, MD (US); Haley Huang, Baltimore, MD (US); Adam Clark, Baltimore, MD (US)

(21) Appl. No.: 13/875,496

(22) Filed: May 2, 2013

Related U.S. Application Data
(60) Provisional application No. 61/641,411, filed on May 2, 2012.

(51) Int. Cl. A61B 17/04

(52) U.S. Cl. CPC A61B 17/0491
USPC 606/144

(57) ABSTRACT

An embodiment in accordance with the present invention provides a device and method for suturing the fascia or other tissue in a patient. The device includes a first arm and a second arm pivotally coupled to one another and housing a spring-based lever system that is configured to frictionally hold a portion of a needle within each of the first and second arms. A switch allows the spring-based lever system to be used to pass the needle between the first and second arms. When the switch is in a first position, the needle is frictionally held by the spring-based lever system within the first arm and released within the second arm. Likewise, when the switch is in a second position, the needle is frictionally held by the spring-based lever system within the second arm and released within the first arm.

### CROSS REFERENCE TO RELATED APPLICATIONS

This application claims the benefit of U.S. Provisional Patent Application No. 61/641,411 filed on May 2, 2012, which is incorporated by reference, herein, in its entirety.

### FIELD OF THE INVENTION

The present invention relates generally to surgery. More particularly, the present invention relates to a device for suturing closed a surgical incision.

### BACKGROUND OF THE INVENTION

Approximately 4-5 million abdominal surgeries are performed in the United States each year. Of those abdominal surgeries, approximately 9%-19% have preventable complications such as hernia formation, wound dehiscence, infections, and organ evisceration, which generate an estimated $2.5 billion dollars in health care costs annually. Many of these complications can at least in part, be caused by an improper closure of the fascia layer that is a part of the muscle and protects inner organs. In general, surgeons manually stitch the incised fascia together with a large, semi-circular needle attached to suture, and often use extraneous devices such as a malleable, which is a metal plate that is placed between the internal organs and the fascia to prevent bowel puncture. Best practices procedures call for sutures to close fascia to be placed evenly 1 cm apart from each other, in order to prevent complications. However, this is difficult and time consuming, which can lead to inconsistencies in suture placement.

It would therefore be advantageous to provide a new device and method for suturing fascia that allows for precision sutures having the appropriate spacing and tension in a shorter amount of time.

### SUMMARY OF THE INVENTION

The foregoing needs are met, to a great extent, by the present invention, wherein in one aspect a device for suturing includes a first arm having a distal end and a proximal end, and the device having a second arm having a distal end and a proximal end, wherein the second arm is configured to interlock with the first arm. A needle is releasably disposed at least partially within a first chamber defined by the first arm. The device includes a spring-based lever system disposed such that said spring-based lever system frictionally holds a first portion of the needle in the first chamber. Additionally, the device includes a switch coupled to the spring-based lever system. The switch has a first position and a second position, and the switch is configured to engage the spring-based lever system, such that when the switch is in the first position the first portion of the needle is held within the first chamber. When the switch is in the second position the first portion of the needle is released from the first chamber.

In accordance with another aspect of the present invention, the first and second arms are configured such that when the proximal end of the first arm and the proximal end of the second arm are compressed together the distal end of the first arm and the distal end of the second arm are also compressed together. The second arm further includes a second chamber defined by the second arm. The second chamber is configured to receive a second portion of the needle, when the distal end of the first arm and the distal end of the second arm are compressed together. The switch is further configured to engage the spring-based lever system, such that when the switch is in the second position the second portion of the needle is frictionally held within the second chamber and when the switch is in the first position the second portion of the needle is released from the second chamber.

In accordance with still another aspect of the present invention, the device can include a calibration system to aid surgeons in suture placement. More specifically, the calibration system can include a circular guide with a radius corresponding to a distance between adjacent sutures. A gauge can be used to measure a force applied to the device. Additionally, the device can include a meter that measures a tension of a suture placement created using the device. The tension is measured by securing a string of the suture within a loop of the meter that measures the tension of the suture, and markers can indicate an acceptable or unacceptable range of tension for the suture. The first end of the first arm and the first end of the second arm can include movable jaws, such that the jaws are adjustable to allow for different penetration depths of a tissue to be sutured. Also, the second end of the first arm and the second end of the second arm can further include movable handles configured to swivel and allow for different wrist orientations during a suturing procedure.

In accordance with yet another aspect of the present invention, a method for suturing fascia in a patient includes puncturing the fascia with a first side of a double ended needle. The double ended needle is attached to a length of suture. The method includes compressing a first arm and a second arm of a suturing device together. The first arm of the suturing device releasably holds a second end of the double ended needle, such that the second arm of the suturing device comes to hold the first end of the double ended needle. Additionally, the method includes toggling a switch coupled to a spring based lever system, such that the spring based lever system releases the second end of the needle and grasps the first end of the needle, and pulling the double ended needle and at least a portion of the attached length of suture through the fascia.

In accordance with another aspect of the present invention, the method further includes measuring tension in the length of suture, using a tension meter. The first arm and the second arm of the suturing device can be adjusted in order to allow for different penetration depths of the fascia to be sutured. The method can also include passing the double ended needle between a first chamber in the first arm and a second chamber in the second arm. A first component of the spring based lever system can be configured to hold an end of the double ended needle being disposed in the first chamber in the first arm. A second component of the spring based lever system, also can be configured to hold an end of the double ended needle, being disposed in the second chamber of the second arm. The method can also include using a calibration system on the suturing device to determine a distance between sutures. The calibration system further includes a circular guide with a radius corresponding to the distance between sutures. In addition, the method includes measuring the force applied to the suturing device with a gauge coupled to the suturing device.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a perspective view of a suturing device according to an embodiment of the present invention.

FIG. 2 illustrates a sectional view of a suturing device according to an embodiment of the present invention.

FIG. 3 illustrates a sectional view of the suturing device according to the embodiment of FIG. 2.

FIG. 4 illustrates an exploded view of a suturing device according to an embodiment of the present invention.

FIG. 5 illustrates a sectional view of a suturing device according to an embodiment of the present invention being used in a suturing procedure.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a device and method for suturing the fascia or other tissue in a patient. The device includes a first arm and a second arm pivotally coupled to one another and housing a spring-based lever system that is configured to frictionally hold a portion of a needle within each of the first and second arms. A switch allows the spring-based lever system to be used to pass the needle between the first and second arms. When the switch is in a first position, the needle is frictionally held by the spring-based lever system within the first arm and released within the second arm. The chamber also holds the needle to a defined curve geometry to secure appropriate clamp orientation. Likewise, when the switch is in a second position, the needle is frictionally held by the spring-based lever system within the second arm and released within the first arm.

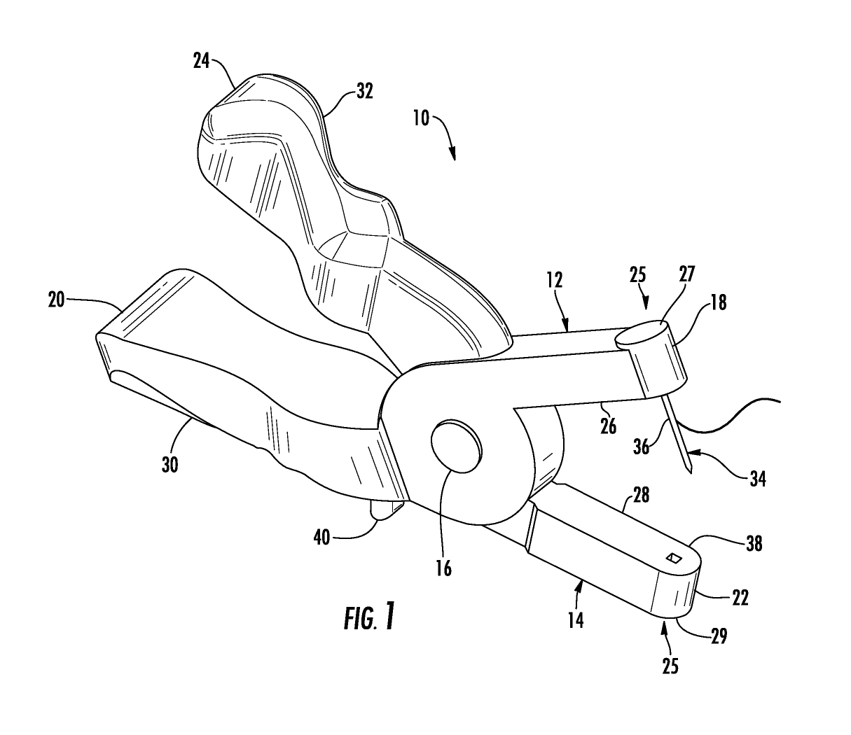

FIG. 1 illustrates a perspective view of a suturing device according to an embodiment of the present invention. As illustrated in FIG. 1 the device 10 includes a first arm 12 and a second arm 14. The first arm 12 and the second arm 14 are coupled to one another with bolt 16 in an interlocked fashion, such that the first arm 12 and the second arm 14 pivot relative to one another about bolt 16. The first arm 12 includes a distal end 18 and a proximal end 20 and the second arm 14 includes a distal end 22 and a proximal end 24. The first arm 12 can include a first arm jaw 26 positioned near the distal end 18, and the second arm 14 can include a second arm jaw 28 positioned near the distal end 22. The first and second arm jaws 26 and 28 can also be adjustable, to allow for sutures to be placed at different depths along the patient's fascia or tissue. In addition, the first and second arm jaws 26 and 28 can be removable and replaceable to accommodate different suture depths as well as patients of different sizes.

The first and second arm jaws 26 and 28 can include a calibration device 25, to assist the physician in placement of the sutures in the tissue. As illustrated in FIG. 1, the calibration device takes the form of a first circular guide 27 and a second circular guide 29. The first and second circular guides 27 and 29 have a radius that corresponds to a desired distance between sutures for a given procedure. For example, in the suturing of fascia it is preferable for sutures to be placed at 1 cm increments. Therefore, for such an application, the circular guide could have a radius of 1 cm. However, this example is not meant to be limiting and other radii could be used for devices configured for other surgical suturing procedures known to those of skill in the art. Additionally, the radius of the first and second circular guides 27 and 29 could also be adjustable to allow one device to indicate different suture spacing.

Further, as illustrated in FIG. 1, the first arm 12 can include a first handle 30 positioned near the proximal end 20, and the second arm 14 can include a second handle 32 positioned near the proximal end 24. The first and second handles 30 and 32 can be configured to have an ergonomic design, as illustrated in FIG. 1, in order to enhance physician comfort and control in using the device. Additionally, a portion of the first and second handles 30 and 32 can be configured to rotate about an axis of the first arm 12 and the second arm 14. This would allow for a physician to assume different wrist orientations during a procedure. The first and second handles 30 and 32 are squeezed together to pivot the first and second arms 12, 14 about the bolt 16, such that the first arm jaw 26 and the second arm jaw 28 are brought into closer proximity.

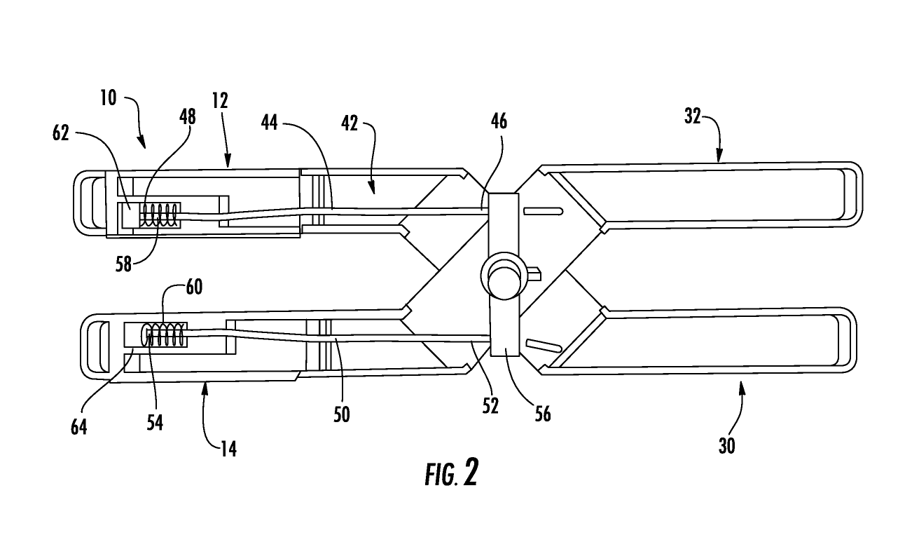

The device 10 also includes a needle 34. Although not illustrated in FIG. 1, the needle 34 can be tapered on both its first end and its second end, such that both the first end and the second end are able to pierce the tissue to be sutured. As illustrated in FIG. 1, the needle 34 has a suture secured to it near approximately a mid-point 36 of the needle 34. The needle 34 can be passed between the first arm jaw 26 and the second arm jaw 28. FIG. 1 illustrates the needle 34 being held within a first chamber (not illustrated), defined by the first arm jaw 26. The second arm jaw 28 also defines a second chamber 38, having the same structure as the first chamber. When the first arm jaw 26 and the second arm jaw 28 are brought into proximity by compressing the first and second handles 30, 32 together, the needle 34 is inserted into the second chamber 38. Switch 40 is then used to secure the needle 34 within the second chamber 38 and release it from the first chamber. Alternately, however, a built-in, self-actuating, mechanism that allows the needle to be passed automatically without the use of a switch could also be used, as well as any other suitable mechanism for causing the needle to be passed back and forth between the first chamber and the second chamber that is known to one of skill in the art. The mechanism for securing and releasing the needle 34 will be described in further detail below with respect to FIGS. 2, 3, and 4.

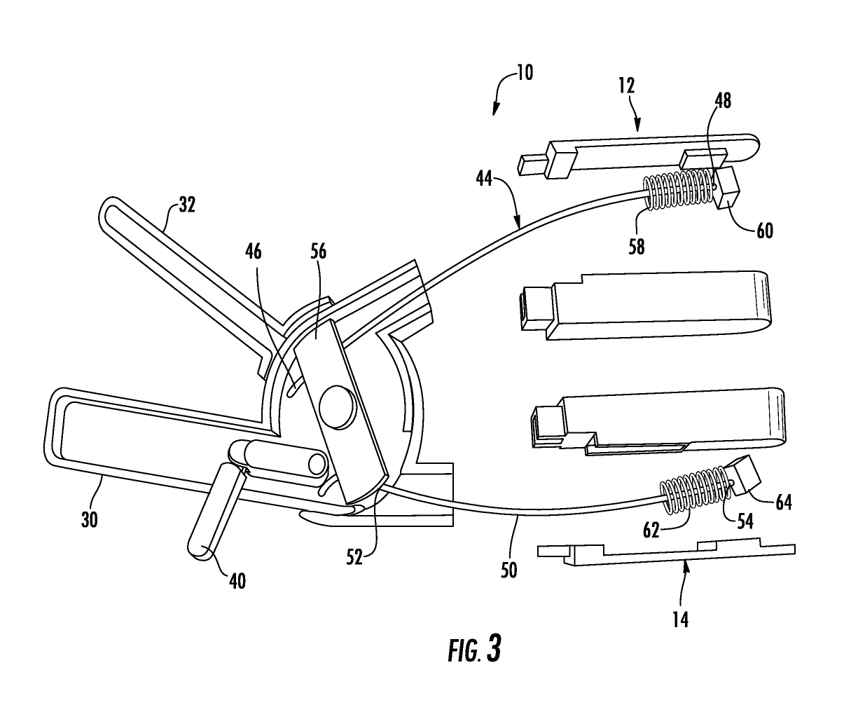

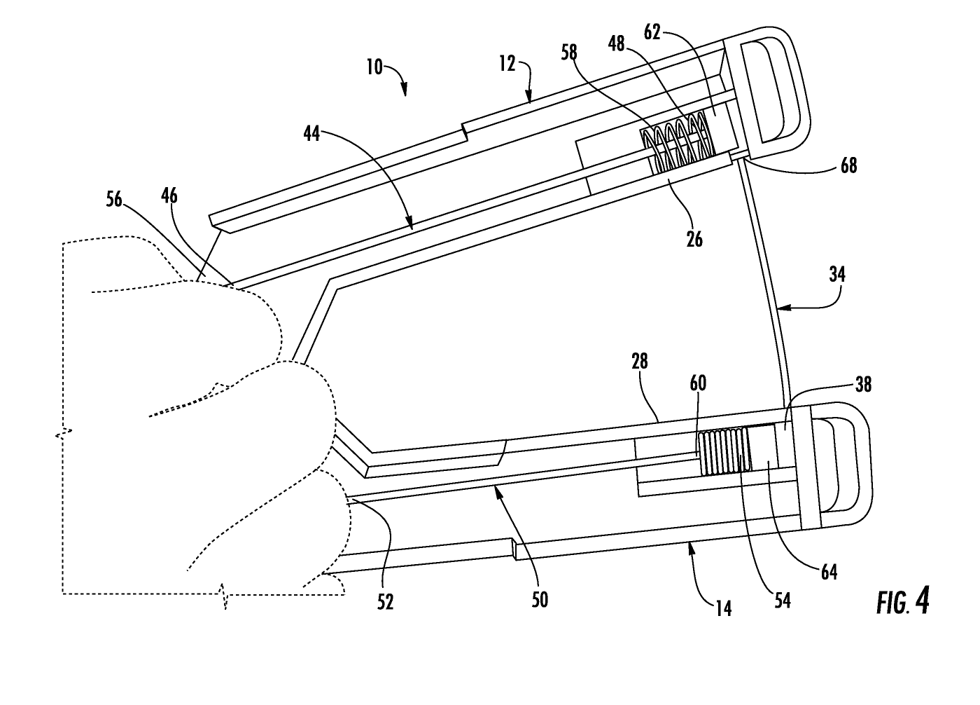

FIG. 2 illustrates a sectional view of a suturing device according to an embodiment of the present invention, and FIG. 3 illustrates an exploded view of a suturing device according to an embodiment of the present invention. As illustrated in FIGS. 2 and 3, the device 10 includes a spring-based lever system 42 disposed within the first arm 12 and the second arm 14. The spring-based lever system 42 includes a first arm wire 44 having a proximal end 46 and a distal end 48 and a second arm wire 50 having a proximal end 52 and a distal end 54. The proximal ends 44 and 52 of the wires 44 and 50 are coupled to a toggle 56. The distal ends 48 and 52 of the wires 44 and 50 are coupled to a first arm spring 58 and a second arm spring 60, respectively. The first and second arm springs 58 and 60, in turn, are further coupled to a first arm grip 62 and a second arm grip 64, respectively. As illustrated in FIG. 3, the toggle 56 is coupled to the switch 40. The switch 40 has a first position and a second position. When the switch 40 is moved between the first and second positions, the switch 40 then moves the toggle 56, which moves the wires 44 and 50. When one of the wires 44 or 50 is pulled proximally, it applies a pressure to the corresponding spring 58 or 60, and pulls back on corresponding grip 62 or 64. This motion allows for the needle (not shown) to be gripped and released, as will be discussed further with respect to FIG. 4. While a spring-based lever system is described herein, it is not meant to be limiting and any system for gripping and releasing known to one of skill in the art could be used.

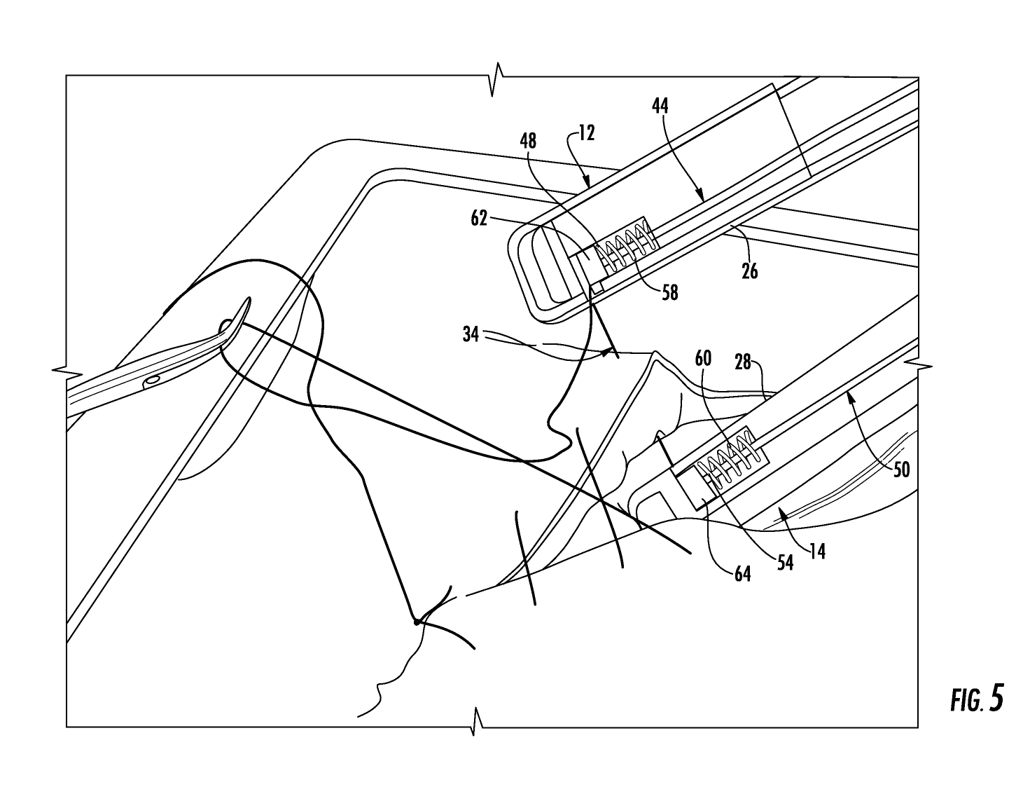

FIG. 4 illustrates a sectional view of the suturing device according to an embodiment of the present invention, and FIG. 5 illustrates a sectional view of a suturing device according to an embodiment of the present invention being used in a suturing procedure. As illustrated in FIG. 4, when toggle 56 is moved to the first position using the switch, first arm grip 62 engages a portion of needle 34 within the first arm chamber 68. If the first arm jaw 26 and the second arm jaw 28 are compressed close to one another, a portion of the needle 34 is received in the second arm chamber 38. When the toggle 56 is moved to the second position, the needle 34 is released from the first arm chamber 68 and gripped in the second arm chamber 38. It is in this way that the needle 34 is passed between the first arm jaw 26 and the second arm jaw 28 and can be used in a suturing procedure, as illustrated in FIG. 5. With respect to FIG. 5, in use, the surgeon places the fascial layer 70 or tissue to be sutured between the first arm jaw 26 and the second arm jaw 28 of the device 10, loaded with needle 34 secured in the first arm chamber 68 or the second arm chamber 38. The surgeon then squeezes the handles, which close the first arm jaw 26 and second arm jaw 28 together. The surgeon toggles the switch that would cause the needle 34 to be released from the chamber 38 or 68 that holds it. At the same time, the toggle 56 would also cause the needle 34 to be secured in the chamber opposite the one it was previously held within. The surgeon releases the handles and the needle 34 will have passed from one of the jaws through the fascia 70, to the opposing jaw. The process is repeated until the fascial layer 70 is closed.

Additional features such as a tension meter and a force gauge can be included on the device. For instance, the tension meter can work by securing the suture string within a tension measuring loop of the tension meter. Markers can then indicate whether the tension of the suture is acceptable or unacceptable. The force being applied to the device can also be measured with a force gauge incorporated into the device. The device can also include an automatic clamping feature and a light that casts a shadow to direct the physician where to place the next suture. The needle can be coupled to a spool mechanism to hold suture and keep it out of the way and the device could also include a suture and needle loading mechanism or cartridge to provide ease of loading and replacing the needle and suture.

While this system has been described for use in suturing fascia, it need not be limited to this application and could be used for suturing other tissues such as tendon, bowel, and vascular tissue, or any other suitable application or variation known to one of skill in the art. The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A device for suturing comprising:
a first arm having a distal end and a proximal end;
a second arm having a distal end and a proximal end, wherein the second arm is configured to interlock with the first arm;
a needle releasably disposed at least partially within a first chamber defined by the first arm;
a spring-based lever system disposed such that said spring-based lever system frictionally holds a first portion of the needle in the first chamber; and
a switch coupled to the spring-based lever system, said switch having a first position and a second position and said switch being configured to engage the spring-based lever system, such that when the switch is in the first position, the first portion of the needle is held within the first chamber and when the switch is in the second position, the first portion of the needle is released from the first chamber.

2. The device of claim 1 wherein the first and second arms are configured such that when the proximal end of the first arm and the proximal end of the second arm are compressed together the distal end of the first arm and the distal end of the second arm are also compressed together.

3. The device of claim 2 wherein the second arm further comprises a second chamber defined by the second arm and wherein the second chamber is configured to receive a second portion of the needle when the distal end of the first arm and the distal end of the second arm are compressed together.

4. The device of claim 3 wherein the switch is further configured to engage the spring-based lever system such that when the switch is in the second position the second portion of the needle is frictionally held within the second chamber and when the switch is in the first position the second portion of the needle is released from the second chamber.

5. The device of claim 1 further comprising a calibration system to aid surgeons in suture placement.

6. The device of claim 5 wherein the calibration system comprises a circular guide with a radius corresponding to a distance between sutures.

7. The device of claim 1 further comprising a gauge that measures a force applied to the device.

8. The device of claim 1 further comprising a meter that measures a tension of a suture created using the device.

9. The device of claim 8 wherein the tension is measured by securing a string of the suture within a loop of the meter that measures the tension of the suture.

10. The device of claim 9 further comprising markers that indicate an acceptable or unacceptable range of tension for the suture.

11. The device of claim 1 wherein the first end of the first arm and the first end of the second arm comprise movable jaws, such that the jaws are adjustable to allow for different penetration depths of a tissue to be sutured.

12. The device of claim 1 wherein the second end of the first arm and the second end of the second arm comprise movable handles configured to swivel and allow for different wrist orientations during a suturing procedure.

13. A method for suturing fascia in a patient comprising:
puncturing the fascia with a first side of a double ended needle, wherein said double ended needle is attached to a length of suture;
compressing a first arm and a second arm of a suturing device together, wherein the first arm of the suturing device releasably holds a second end of the double ended needle, such that the second arm of the suturing device comes to hold the first end of the double ended needle;
toggling a switch coupled to a spring based lever system, such that the spring based lever system releases the second end of the needle and grasps the first end of the needle; and
pulling the double ended needle and at least a portion of the attached length of suture through the fascia.

14. The method of claim 13 further comprising measuring tension in the length of suture, using a tension meter.

15. The method of claim 13 further comprising adjusting the first arm and the second arm of the suturing device in order to allow for different penetration depths of the fascia to be sutured.

16. The method of claim 13 further comprising passing the double ended needle between a first chamber in the first arm and a second chamber in the second arm.

17. The method of claim 16 further comprising a first component of the spring based lever system configured to hold an end of the double ended needle being disposed in the first chamber in the first arm and a second component of the spring based lever system, also configured to hold an end of the double ended needle, being disposed in the second chamber of the second arm.

18. The method of claim 13 further comprising using a calibration system on the suturing device to determine a distance between sutures.

19. The method of claim 18 wherein the calibration system further comprises a circular guide with a radius corresponding to the distance between sutures.

20. The method of claim 13 further comprising measuring the force applied to the suturing device with a gauge coupled to the suturing device.
